(19) United States
(12) Patent Application Publication
(10) Pub. No.: US 2026/0227540 A1
(43) Pub. Date: Aug. 6, 2026

(54) GRADIENT RESONANCE IN SURFACE ELECTROMAGNETIC WAVE PROPAGATION FOR COMMUNICATION, SENSING, AND IMAGING

(71) Applicant: Saltenna Inc., McLean, VA (US)

(72) Inventors: Igor SMOLYANINOV, Columbia, MD (US); Quirino BALZANO, Annapolis, MD (US)

(21) Appl. No.: 19/134,613

(22) PCT Filed: Feb. 6, 2025
(86) PCT No.: PCT/US2025/014746

§ 371 Date: May 30, 2025

(51) Int. Cl. G01V 3/14; A61B 5/00

(52) U.S. Cl. CPC G01V 3/14; G01N 21/553; A61B 5/0042

(57) ABSTRACT

Methods, systems, and devices for communication, sensing, and imaging based on gradient resonance in surface electromagnetic waves are described. In some examples, a gradient of a dielectric permittivity of a medium may be obtained along a propagation path of surface electromagnetic waves. An effective potential may be determined based on the gradient and a second derivative of the dielectric permittivity. A resonance condition may be identified when the real part of the dielectric permittivity squared approaches zero. Surface electromagnetic waves may be generated in response to the identified resonance condition, and a gradient resonance in the surface electromagnetic waves may be detected. These techniques may enable enhanced communication, sensing, and imaging applications, including underwater communication, agricultural monitoring, and bio-imaging, by leveraging the resonance properties of surface electromagnetic waves.

### FIELD OF TECHNOLOGY

The present disclosure relates generally to database systems and data processing, and more specifically to gradient resonance in surface electromagnetic wave propagation for communication, sensing, and imaging.

### BACKGROUND

Electromagnetic wave-based technologies have long been utilized in diverse applications, including communication, imaging, and sensing. These technologies play a critical role in fields such as biomedical imaging, where precise visualization of internal structures is essential, and in agriculture, where monitoring crop health and water content is vital for optimizing yield. The ability to enhance resolution, sensitivity, and range in these applications continues to drive innovation across industries, from healthcare to environmental monitoring and beyond.

### SUMMARY

The described techniques relate to improved methods, systems, devices, and apparatuses that support techniques for gradient resonance in surface electromagnetic wave propagation for communication, sensing, and imaging. In some examples, the method may utilize gradient resonance in surface electromagnetic wave propagation, where the dielectric permittivity of the medium may vary gradually. This approach may leverage the resonance conditions when the real part of the permittivity squared is close to zero, significantly enhancing the propagation characteristics. By transforming Maxwell's equations into a form analogous to the Schrödinger equation, some implementations may identify the effective potential seen by photons, which may include terms proportional to the gradients and second derivatives of the dielectric permittivity. The primary term, proportional to the square of the first derivative of permittivity, may exhibit pronounced resonance behavior under specific conditions.

This gradient resonance, referred to as plasmonic gradient resonance (PGR), may be experimentally observed in various media, including water with different salinity levels. Some implementations may demonstrate that under PGR conditions, the communication distance, detection sensitivity, and imaging resolution are significantly improved. Applications of this method may extend to underwater communication, biomedical imaging, and agricultural sensing, among others. The ability to achieve high sensitivity and resolution without the need for large magnetic fields, as required in Nuclear Magnetic Resonance (NMR), may position some implementations as a groundbreaking advancement in the field of surface electromagnetic wave technologies.

A method for communication, sensing, and imaging based on gradient resonance in surface electromagnetic waves is described. The method may include obtaining a gradient of a dielectric permittivity of a medium along a propagation path of surface electromagnetic waves. The method may include obtaining an effective (e.g., maximum productive) potential based on the gradient of the dielectric permittivity and a second derivative of the dielectric permittivity. The method may include identifying a resonance condition in response to a real part of the dielectric permittivity squared approaching zero. The method may include generating surface electromagnetic waves in response to the identified resonance condition. The method may include detecting a gradient resonance in the surface electromagnetic waves.

A system configured for communication, sensing, and imaging based on gradient resonance in surface electromagnetic waves is described. The system may include a processor. The system may include memory coupled with the processor. The system may include instructions stored in the memory and executable by the processor to cause the system to obtain a gradient of a dielectric permittivity of a medium along a propagation path of surface electromagnetic waves. The system may include instructions to obtain an effective potential based on the gradient of the dielectric permittivity and a second derivative of the dielectric permittivity. The system may include instructions to identify a resonance condition in response to a real part of the dielectric permittivity squared approaching zero. The system may include instructions to generate surface electromagnetic waves in response to the identified resonance condition. The system may include instructions to detect a gradient resonance in the surface electromagnetic waves.

Another system for communication, sensing, and imaging based on gradient resonance in surface electromagnetic waves is described. The system may include means for obtaining a gradient of a dielectric permittivity of a medium along a propagation path of surface electromagnetic waves. The system may include means for obtaining an effective potential based on the gradient of the dielectric permittivity and a second derivative of the dielectric permittivity. The system may include means for identifying a resonance condition in response to a real part of the dielectric permittivity squared approaching zero. The system may include means for generating surface electromagnetic waves in response to the identified resonance condition. The system may include means for detecting a gradient resonance in the surface electromagnetic waves.

A non-transitory computer-readable medium storing code for communication, sensing, and imaging based on gradient resonance in surface electromagnetic waves is described. The code may include instructions executable by a processor to perform communication, sensing, and imaging based on gradient resonance in surface electromagnetic waves. The code may include instructions executable by a processor to obtain a gradient of a dielectric permittivity of a medium along a propagation path of surface electromagnetic waves. The code may include instructions executable by a processor to obtain an effective potential based on the gradient of the dielectric permittivity and a second derivative of the dielectric permittivity. The code may include instructions executable by a processor to identify a resonance condition in response to a real part of the dielectric permittivity squared approaching zero. The code may include instructions executable by a processor to generate surface electromagnetic waves in response to the identified resonance condition. The code may include instructions executable by a processor to detect a gradient resonance in the surface electromagnetic waves.

Some examples of the method, systems, and non-transitory computer-readable medium described herein may further include operations, features, means, or instructions for generating surface electromagnetic waves at a frequency corresponding to the identified resonance condition.

Some examples of the method, systems, and non-transitory computer-readable medium described herein may further include operations, features, means, or instructions for adapting the dielectric permittivity of the medium to achieve the gradient of the dielectric permittivity along the propagation path.

Some examples of the method, systems, and non-transitory computer-readable medium described herein may further include operations, features, means, or instructions for measuring the dielectric properties of the medium to determine the gradient of the dielectric permittivity.

Some examples of the method, systems, and non-transitory computer-readable medium described herein may further include operations, features, means, or instructions for analyzing the detected gradient resonance to determine the communication distance of the surface electromagnetic waves.

Some examples of the method, systems, and non-transitory computer-readable medium described herein may further include operations, features, means, or instructions for using the detected gradient resonance to perform imaging of biological tissues.

In some examples of the method, systems, and non-transitory computer-readable medium described herein, the gradient of the dielectric permittivity may be obtained by varying the salinity of water in the medium.

In some examples of the method, systems, and non-transitory computer-readable medium described herein, the effective potential may be determined by the first derivative of the dielectric permittivity along the propagation path.

In some examples of the method, systems, and non-transitory computer-readable medium described herein, the surface electromagnetic waves may be generated at a frequency range between 1 MHz and 1000 MHz.

Some examples of the method, systems, and non-transitory computer-readable medium described herein may further include operations, features, means, or instructions for using the detected gradient resonance to enhance the sensitivity of RF imaging techniques.

In some examples of the method, systems, and non-transitory computer-readable medium described herein, the surface electromagnetic waves may be used to detect the presence of underground water beneath agricultural fields.

In some examples of the method, systems, and non-transitory computer-readable medium described herein, the gradient resonance may be identified in response to a change in the dielectric properties of the medium.

### BRIEF DESCRIPTION OF THE DRAWINGS

FIG. 1 illustrates an example of a system for data processing that supports gradient resonance in surface electromagnetic wave propagation for communication, sensing, and imaging in accordance with aspects of the present disclosure.

FIG. 2 shows dielectric interface illustration which supports techniques for gradient resonance in surface electromagnetic wave propagation for communication, sensing, and imaging in accordance with various aspects of the present disclosure.

FIG. 3 shows a dielectric properties graph illustrating the real and imaginary parts of the dielectric permittivity of water at three different salinity levels in accordance with various aspects of the present disclosure.

FIG. 4 shows a line graph illustrating the propagation distance to the skin depth ratio for surface electromagnetic waves at the pool water-air interface in accordance with various aspects of the present disclosure.

FIG. 5 shows modeling of surface electromagnetic wave excitation at a boundary between the bone tissue and the gray matter in a human brane, while the excitation source was placed at different distances from the boundary in accordance with various aspects of the present disclosure.

FIG. 6 shows forest canopy transition which supports techniques for gradient resonance in surface electromagnetic wave propagation for communication, sensing, and imaging in accordance with various aspects of the present disclosure.

FIG. 7 shows a block diagram of an apparatus that detects gradient resonance in surface electromagnetic wave propagation for communication, sensing, and imaging in accordance with various aspects of the present disclosure.

FIG. 8 shows a block diagram of a gradient resonance detection component that supports gradient resonance in surface electromagnetic wave propagation for communication, sensing, and imaging in accordance with various aspects of the present disclosure.

FIG. 9 shows a diagram of a system including a device that supports gradient resonance in surface electromagnetic wave propagation for communication, sensing, and imaging in accordance with various aspects of the present disclosure.

FIGS. 10 and 11 show flowcharts illustrating methods that support gradient resonance in surface electromagnetic wave propagation for communication, sensing, and imaging in accordance with various aspects of the present disclosure.

### DETAILED DESCRIPTION

Methods, systems, devices, and apparatuses that support techniques for gradient resonance in surface electromagnetic wave propagation for communication, sensing, and imaging are disclosed. In some examples, despite promising theoretical advancements in gradient-index materials for surface electromagnetic wave propagation, practical implementations have faced significant challenges. Traditional methods that rely on sharp interfaces do not fully leverage the potential of gradual changes in dielectric permittivity, resulting in suboptimal performance in communication, sensing, and imaging applications. The lack of a clear understanding and utilization of resonance conditions, where the real part of the permittivity squared approaches zero, has further hindered the development of more effective techniques. Consequently, there is a need for a method that can harness these resonance conditions to significantly enhance the propagation characteristics of surface electromagnetic waves, thereby improving the efficiency and sensitivity of various applications.

According to some implementations, gradient resonance may be utilized in surface electromagnetic wave propagation, where the dielectric permittivity of the medium may vary gradually along the z-coordinate, which is orthogonal to the gradient interface. This may achieve resonance conditions when the real part of the permittivity squared may be close to zero, potentially enhancing the propagation characteristics of surface electromagnetic waves.

The theoretical framework may involve the macroscopic Maxwell equations in a non-magnetic medium, where the dielectric permittivity may depend only on the z-coordinate. The dielectric permittivity may be expressed as a complex function with real and imaginary parts. The Maxwell equations may be transformed into a wave equation, which may coincide with the form of the Schrödinger equation. For transverse electric polarization, the effective potential energy may not support surface wave solutions. However, for transverse magnetic polarization, the effective potential energy may support plasmon-like surface electromagnetic wave solutions. The effective potential may be dominated by a term proportional to the square of the gradient of the dielectric permittivity, which may exhibit pronounced resonance behavior near the point where the real part of the permittivity squared may pass through zero. This situation may be referred to as plasmonic gradient resonance or PGR.

Experimental observations may include gradient resonance in surface wave propagation along the water-air interface. The dielectric properties of water at different salinity levels may be measured, and the gradient resonance conditions may be observed around a specific resonant frequency in pool water. Underwater radio communication experiments may demonstrate clear resonance behavior in communication distance as a function of frequency. The ratio of communication distance to skin depth may exhibit a pronounced resonance peak.

Applications of PGR may include extending communication range, as demonstrated in underwater experiments. Some implementations may be used for mine and foreign object detection on water surfaces. Biomedical imaging applications may be contemplated, with potential resolution and contrast improvements comparable to nuclear magnetic resonance (NMR) but without the need for large magnetic fields. Some implementations may be applied in jungle communication and agricultural imaging. Surface waves may propagate along the surface of plants, enabling remote sensing of crop health and underground water detection. A hyperspectral remote radio frequency imaging scheme may be implemented, measuring the radio frequency spectrum at several frequencies to create a composite map of the land.

The mathematical and numerical analysis may involve the effective Schrödinger equation for transverse magnetic polarization, analyzed numerically and analytically for simple spatial distributions of the dielectric permittivity. The effective potential may be described by a specific equation, leading to plasmon-like solutions with resonance properties. The resonance behavior may be dominated by the term proportional to the square of the gradient of the dielectric permittivity. Numerical simulations may show surface electromagnetic wave propagation along interfaces between different tissues, such as bone and gray matter in the human body.

Some implementations may offer potential improvements in imaging and sensing resolution. Some implementations may be sensitive to small variations in tissue composition, making it suitable for detecting conditions like cancer. Some implementations may be used for bio-imaging and bio-sensing applications, leveraging the water content in human tissues.

The propagation characteristics of surface electromagnetic waves may be enhanced under PGR conditions. The communication distance to skin depth ratio may be significantly increased, indicating improved propagation efficiency. The system may achieve high sensitivity and resolution in various applications, including medical imaging and agricultural sensing.

Some implementations may be used for hyperspectral radio frequency imaging, measuring the radio frequency spectrum at multiple frequencies. This approach may enable the creation of detailed composite maps for applications like crop health monitoring and underground water detection.

Aspects of the subject matter described in this disclosure can be implemented to realize one or more of the following potential advantages. The described techniques may be implemented to support enhanced communication capabilities in challenging environments, such as underwater or dense foliage, by leveraging the gradient resonance conditions. The system may provide improved sensitivity and accuracy in detecting and imaging various materials and biological tissues, potentially leading to advancements in medical diagnostics and agricultural monitoring. The ability to achieve resonance at specific frequencies may enable precise control over the propagation characteristics of surface electromagnetic waves, which may result in more efficient and effective sensing and imaging applications. The use of gradient resonance may allow for the development of compact and portable devices capable of high-resolution imaging without the need for large and complex equipment. The described methods may facilitate the creation of detailed hyperspectral maps, which may be valuable for environmental monitoring and resource management.

Aspects of the disclosure are initially described in the context of networked computing systems. Aspects of the disclosure are additionally illustrated by and described with reference to example implementations. Aspects of the disclosure are further illustrated by and described with reference to apparatus diagrams, system diagrams, and flowcharts that relate to gradient resonance in surface electromagnetic wave propagation for communication, sensing, and imaging.

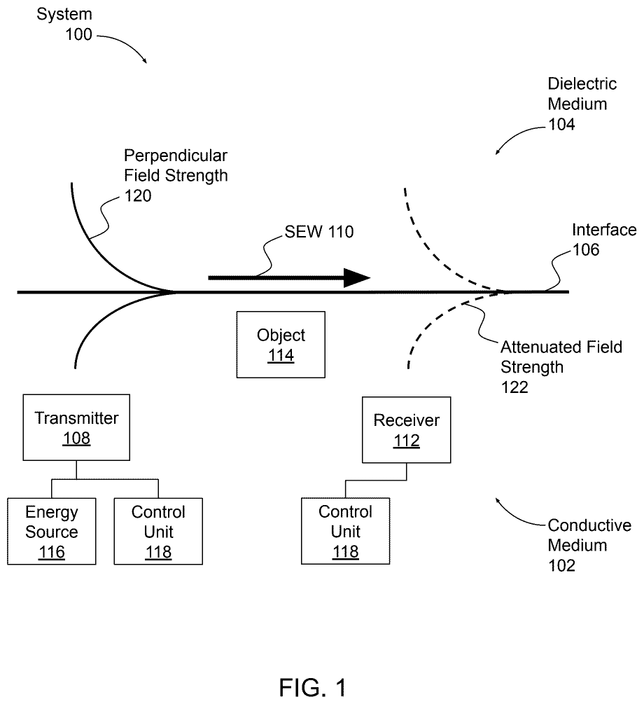

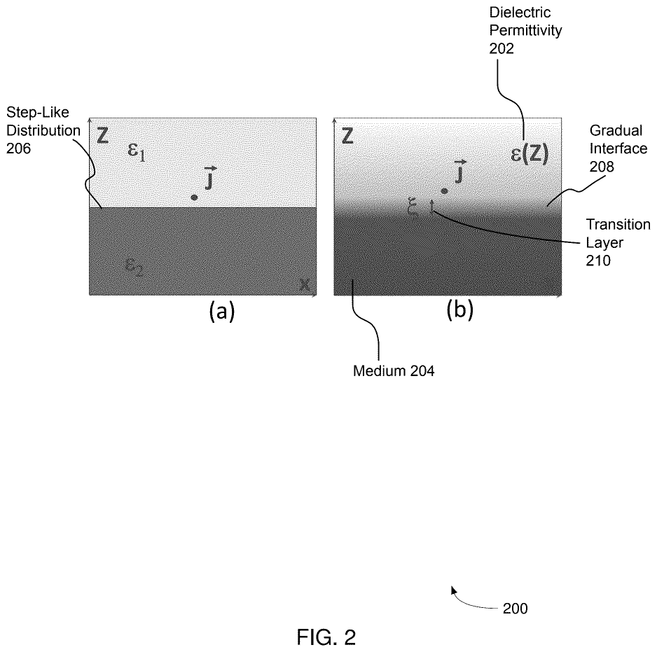

FIG. 1 illustrates a system 100 that demonstrates the concept of surface electromagnetic waves (SEWs), in accordance with one or more implementations. The system 100 may include a conductive medium 102, such as a body of water, organic tissue, a metallic plane, and/or other conductive media. Adjacent to the conductive medium 102, there may be a dielectric medium 104, such as air and/or other dielectric media, which may interface with the conductive medium 102. The interface 106 between the conductive medium 102 and the dielectric medium 104 may be where SEWs are generated and may propagate. In some implementations, the interface 106 may not be abrupt and, instead, may include a transition layer of finite thickness where the dielectric constant of the media changes in a direction perpendicular to the interface 106 (see, e.g., FIG. 2 part (b)).

The system 100 may also include an antenna 108, which may be positioned near the interface 106. The antenna 108 may be positioned within the conductive medium 102 or within the dielectric medium 104. The antenna 108 may be responsible for generating an electromagnetic field that may excite SEWs at the interface 106 of the conductive medium 102 and the dielectric medium 104. The excited SEWs may then travel along the interface 106, as indicated by the arrow of SEW 110, which may represent the direction of wave propagation.

To help visualize the phenomenon of SEWs, one may consider an analogy to ripples on a pond. When a stone is dropped into a still pond, ripples may form and spread out across the surface of the water. Similarly, the antenna 108 may be thought of as the stone, and the SEWs may be akin to the ripples that spread along the conductive medium 102. Just as the ripples may move outward from the point of impact, SEWs may propagate along the interface 106, carrying energy with them.

The system 100 may further include a detector 112, which may be positioned at a distance from the antenna 108 along the interface 106. The detector 112 may be positioned within the conductive medium 102 or within the dielectric medium 104. The detector 112 may be configured to receive the SEWs after they have propagated along the interface 106. This may be analogous to placing one's hand in the water at a distance from where the stone was dropped, feeling the ripples as they pass by.

Additionally, the system 100 may include an object 114 positioned within the conductive medium 102 or within the dielectric medium 104, which may be representative of an obstacle that SEWs may encounter during propagation. The interaction of SEWs with the object 114 may lead to scattering of waves, similar to how water ripples may change direction or form patterns when they encounter a leaf or a rock in the pond.

The system 100 may include an energy source 116, such as a radio frequency generator, which may be connected to the antenna 108. The energy source 116 may provide the necessary power for the antenna 108 to generate the electromagnetic field that excites the SEWs. This may be thought of as the force with which the stone is thrown into the pond, affecting the size and strength of the resulting ripples.

In some implementations, the system 100 may include a control unit 118, which may be operatively coupled to the antenna 108 and/or the detector 112. The control unit 118 may be responsible for coordinating the generation and detection of SEWs, much like a person orchestrating the timing of stones being dropped into the pond to create a specific pattern of ripples.

From a more technical perspective, SEWs may be understood as a type of wave that propagates along the interface between two media with different dielectric properties. In FIG. 1, the conductive medium 102 and the dielectric medium 104 may form such an interface (e.g., interface 106) where SEWs may be excited and propagate. The antenna 108 may serve as a transducer that converts electrical signals from the energy source 116 into electromagnetic fields, which may then couple to the interface 106 and give rise to SEWs.

The propagation of SEWs along the interface 106 may be characterized by a wave vector that is parallel to the interface 106. This wave vector may be larger than the wave vector of free photons in the dielectric medium 104, which may result in a confinement of the electromagnetic field to the vicinity of the interface 106. The SEW's field strength may decay exponentially in the direction perpendicular to the interface 106, as illustrated by a field strength 120 extending into the dielectric medium 104 and the conductive medium 102. These field strengths may also decay as the SEW propagates along the interface 106, as illustrated by an attenuated field strength 122. The detector 112 may be designed to couple to these confined, attenuated fields and receive the SEWs after they have propagated along the interface 106.

The excitation of SEWs by the antenna 108 may involve the conversion of the electromagnetic energy into a surface-bound mode, which may be facilitated by the specific design of the antenna 108. The antenna 108 may be optimized to match the impedance of the SEWs to maximize energy transfer into the surface wave mode. The object 114 submerged within the conductive medium 102 may introduce perturbations in the SEWs, which may be detected by the detector 112 and analyzed by the control unit 118 to infer properties of the object 114. Examples of such properties may include one or more of size, shape, location, material properties, and/or other properties.

The mathematical description of SEWs may be derived from Maxwell's equations, which govern the behavior of electromagnetic fields. The wave equation for TM-polarized SEWs may be reduced to a one-dimensional Schrödinger equation:

$$\frac{{d}^{2} ψ}{{dz}^{2}} + ( {k}^{2} - V ( z ) ) ψ = 0 \qquad ( EQN . 1 )$$

where ψ is the effective wave function introduced as E_{z}=ψ/√\vec{ϵ}, and V(z) is the effective potential energy that guides the propagation of SEWs along the interface. The term k² may represent the total energy of the SEWs and the term E represents the permittivity of the medium.

For TE-polarized SEWs, the wave equation may not depend on the gradient terms and may be expressed as:

$$\frac{{d}^{2} {E}_{z}}{{dz}^{2}} + {k}^{2} {E}_{z} = 0 \qquad ( EQN . 2 )$$

In the case of a sharp interface between two media with dielectric permittivities ϵ₁ and ϵ₂, the SEW wave vector for TM-polarized waves may be given by:

$${k}_{SEW} = \sqrt{\frac{{ω}^{2}}{{c}^{2}} \frac{{ϵ}_{1} {ϵ}_{2}}{{ϵ}_{1} + {ϵ}_{2}}} \qquad ( EQN . 3 )$$

where ω is the angular frequency of the SEWs, and c is the speed of light in vacuum.

The presence of dielectric permittivity gradients across the interface 106 may lead to additional terms in the effective potential V_{z}, which may result in the formation of a potential well that supports bound states of SEWs. These bound states may correspond to surface modes with long propagation lengths and may be excited by the antenna 108 with appropriate amplitude and phase matching.

The system 100 may thus utilize SEWs for various applications, including communication and sensing, by exploiting the unique properties of SEWs at the interface 106 between the conductive medium 102 and the dielectric medium 104. The control unit 118 may process the received signals to extract information about the propagation and interaction of SEWs with the environment and objects within it.

It should be appreciated by a person skilled in the art that one or more aspects of the disclosure may be implemented in a system to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

FIG. 2 shows dielectric interface 200 illustration which supports techniques for gradient resonance in surface electromagnetic wave propagation for communication, sensing, and imaging in accordance with various aspects of the present disclosure. As depicted in FIG. 2, the pipeline communication diagram 200 may include one or more of a dielectric permittivity 202, a medium 204, a step-like distribution 206, a gradual interface 208, transition layer 210, and/or other components. The dielectric permittivity of the medium ε depends only on z coordinate, which is illustrated by the halftones: In FIG. 2, (a) shows usually considered step-like distribution of ε(z) and (b) shows gradual interface between two media.

The dielectric permittivity 202 may vary depending on the z coordinate. The dielectric permittivity 202 may be a measure of how an electric field affects, and is affected by, a dielectric medium. The dielectric permittivity 202 may have both real and imaginary parts, which may represent the material's ability to store and dissipate electric energy, respectively. In some implementations, the dielectric permittivity 202 may be influenced by factors such as the material composition and environmental conditions like temperature and salinity. For example, in water, the dielectric permittivity 202 may change with varying levels of salinity, as illustrated in the experimental observations.

The medium 204 may be non-magnetic and have a continuous dielectric permittivity. The medium 204 may refer to a substance or material through which electromagnetic waves propagate. The non-magnetic property of the medium 204 may imply that its magnetic permeability is equal to that of free space, meaning it does not significantly affect the magnetic component of electromagnetic waves. The continuous dielectric permittivity of the medium 204 may indicate that the dielectric properties change smoothly without abrupt transitions. In some implementations, the medium 204 may be water, air, or biological tissues, where the dielectric permittivity varies gradually with the z coordinate.

The step-like distribution 206 may illustrate a typical interface between two media. The step-like distribution 206 may represent a sudden change in the dielectric permittivity at the boundary between two different materials. This type of distribution may be common in scenarios where two distinct materials, such as air and water, meet. The step-like distribution 206 may be used to model the behavior of electromagnetic waves at sharp interfaces, where the dielectric properties change abruptly. For example, the interface between air and seawater may exhibit a step-like distribution 206 of dielectric permittivity.

The gradual interface 208 may represent a smooth transition between different media. The gradual interface 208 may occur when the dielectric permittivity changes continuously over a certain distance, rather than abruptly. This smooth transition may be characterized by a gradient in the dielectric properties, which may affect the propagation of electromagnetic waves. The gradual interface 208 may be found in natural environments, such as the transition from the surface of a forest to the open air, where the dielectric properties change gradually due to varying moisture content and vegetation density.

The transition layer 210 may have a specific thickness denoted by ξ. The transition layer 210 may be the region where the dielectric permittivity changes from one value to another over a certain distance. The thickness & of the transition layer 210 may determine the extent of the gradual change in dielectric properties. In some implementations, the transition layer 210 may be found in biological tissues, where the dielectric properties vary smoothly between different types of tissues. For example, the boundary between bone and gray matter in the human brain may have a transition layer 210 with a specific thickness &.

To illustrate the theoretical framework, solutions are considered of the macroscopic Maxwell equations in a geometry in which the medium is non-magnetic (B=H), the dielectric permittivity of a medium is continuous, and it depends only on z coordinate: ε=ε(z)=ε′(z)+iε″(z), as illustrated in FIG. 2 part (b). Under such conditions spatial variables in the Maxwell equations separate, and without the loss of generality we may assume electromagnetic mode propagation in the x direction, leading to field dependencies proportional to e^{i(kx-ωt)}. The macroscopic Maxwell equations may be written as

$$\vec{∇} \cdot \vec{D} = 0 , \vec{∇} \cdot \vec{B} = 0 , \vec{∇} \times \vec{E} = i \frac{ω}{c} \vec{B} , and \vec{∇} \times \vec{B} = - i \frac{ωε}{c} \vec{E} , \qquad ( EQN . 4 )$$

leading to a wave equation

$$\vec{∇} \times ( \vec{∇} \times \vec{E} ) = \frac{{ω}^{2} ε}{{c}^{2}} \vec{E} \qquad ( EQN . 5 )$$

### Since

$$\vec{∇} \times ( \vec{∇} \times \vec{E} ) = - {∇}^{2} \vec{E} + \vec{∇} ( \vec{∇} \cdot \vec{E} ) \qquad ( EQN . 6 )$$

$$\vec{∇} \cdot \vec{E} = - {E}_{z} \frac{{\partial }_{ε} / {\partial }_{z}}{ε} , \qquad ( EQN . 7 )$$

after straightforward transformations we obtain

$$- {∇}^{2} \vec{E} - \vec{∇} ( {E}_{z} \frac{{\partial }_{ε}}{ε \partial z} ) = \frac{{εω}^{2}}{{c}^{2}} \vec{E} \qquad ( EQN . 8 )$$

For the E_{z}=0 (TE) polarization we obtain an effective Schrödinger equation

$$- \frac{{\partial }^{2} {E}_{y}}{{\partial }_{z}^{2}} - \frac{ε ( z ) {ω}^{2}}{{c}^{2}} {E}_{y} = - {k}^{2} {E}_{y} \qquad ( EQN . 9 )$$

while for the E_{z}≠0 (TM) polarization the effective Schrödinger equation is

$$- \frac{{\partial }^{2} {E}_{z}}{{\partial }_{z}^{2}} - \frac{\partial {E}_{z}}{\partial z} \frac{\partial ln ε}{\partial z} - ( \frac{ε ( z ) {ω}^{2}}{{c}^{2}} + \frac{{\partial }^{2} ln ε}{{\partial }_{z}^{2}} ) {E}_{z} = - {k}^{2} {E}_{z} \qquad ( EQN . 10 )$$

In the latter equation the wave function may be introduced as E_{z}=ψ/ε^{1/2}, leading to

$$- \frac{{\partial }^{2} ψ}{{\partial }_{z}^{2}} + ( - \frac{ε ( z ) {ω}^{2}}{{c}^{2}} - \frac{1}{2} \frac{{\partial }^{2} ε}{ε {\partial }_{z}^{2}} + \frac{3}{4} \frac{{( {\partial }_{ε} / {\partial }_{z} )}^{2}}{{ε}^{2}} ) ψ = - \frac{{\partial }^{2} ψ}{{\partial }_{z}^{2}} + V ψ = - {k}^{2} ψ \qquad ( EQN . 11 )$$

For both polarizations-k² plays the role of effective energy in the corresponding Schrödinger equations. Let us study solutions of EQN. 9 and EQN. 11 which have a propagating wave character (Im(k)<<Re(k)).

In the case of TE polarized light (see EQN. 9) the effective potential energy is

$$V ( z ) = - \frac{ε ( z ) {ω}^{2}}{{c}^{2}} ,$$

and there are no surface wave solutions. EQN. 9 only admits propagating solutions described by planar waveguide-like distributions of ε(z), in which the dielectric permittivity is positive and almost pure real. On the other hand, the TM polarized solutions of EQN. 11 are much more interesting. EQN. 11 admits plasmon-like surface electromagnetic wave solutions for a large number of effective potentials described by EQN. 12 below:

$$V ( z ) = - \frac{4 {π}^{7} ε ( z )}{{λ}_{0}^{2}} - \frac{1}{2} \frac{{\partial }^{2} ε}{ε \partial {z}^{7}} + \frac{3}{4} \frac{{( \partial ε / \partial z )}^{2}}{{ε}^{2}} , \qquad ( EQN . 12 )$$

where λ₀ is the free space wavelength. Moreover, unlike conventional plasmonics, according to EQN. 9 a gradient-index medium which is used to support such a propagating surface wave solution does not need to be a low loss medium. For example, a medium having pure imaginary dielectric permittivity ε(z)=iε″(z)=iσ(z)/ε₀ω (where ω is the dielectric permittivity of vacuum, and the medium conductivity σ(z) is expressed in practical SI units) will still result in Im(V)<<Re(V):

$$V = - \frac{i σω}{{ε}_{0} {c}^{2}} - \frac{1}{2} \frac{{\partial }^{2} σ}{σ \partial {z}^{2}} + \frac{3}{4} \frac{{( \partial σ / \partial z )}^{2}}{{σ}^{2}} \qquad ( EQN . 13 )$$

The second and third terms in EQN. 13 are real, and they may become considerably larger than the first term.

In general, solutions of the effective Schrödinger EQN. 11 with an effective potential V(z) given by EQN. 12 must be obtained numerically. However, these equations may be analysed analytically for some simple spatial distributions of ε(z), and it may be demonstrated that plasmon-like solutions of this equation may exhibit pronounced resonance properties.

In particular, in many practical situations the effective potential is dominated by the third term proportional to (dε/dz)², and this leading term exhibits pronounced resonance behaviour near the special point where the real part of ε²=ε′²−ε″² passes through zero. We will refer to this situation as the PGR. Under the PGR conditions, the communication, sensing and imaging properties of surface wave-based RF techniques are strongly enhanced, which is illustrated by our experimental observations described herein.

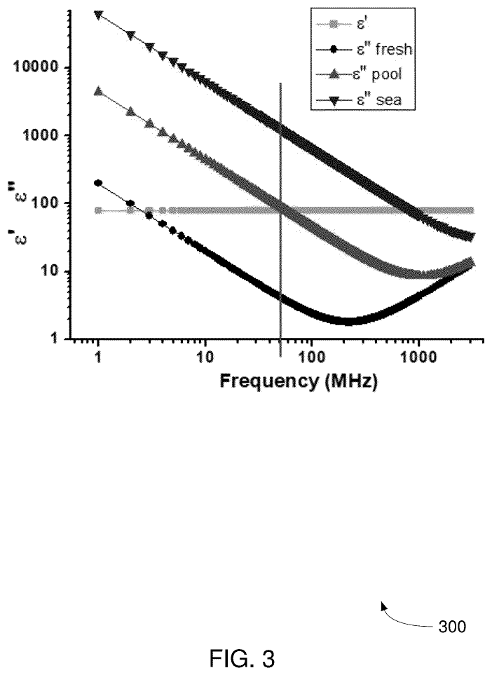

FIG. 3 shows a dielectric properties graph 300 illustrating the real and imaginary parts of the dielectric permittivity of water at three different salinity levels in accordance with various aspects of the present disclosure. The graph 300 may represent the dielectric properties of fresh water, pool water, and seawater across a range of frequencies, which may be relevant to the method for utilizing gradient resonance in surface electromagnetic wave propagation.

The x-axis of the graph 300 may represent the frequency in megahertz (MHz) on a logarithmic scale, ranging from 1 MHz to 1000 MHz. The y-axis may represent the dielectric permittivity, also on a logarithmic scale, ranging from 1 to 10000.

The data points in the graph 300 may be marked with different symbols to differentiate between the real and imaginary parts of the dielectric permittivity for each type of water. The real part of the dielectric permittivity (ε′) may be represented by a solid line, while the imaginary part (ε″) may be represented by various symbols: circles for fresh water, triangles for pool water, and inverted triangles for seawater.

The graph 300 may show that the real part of the dielectric permittivity (ε′) remains relatively constant across the frequency range for all three types of water. In contrast, the imaginary part (ε″) may exhibit a decreasing trend with increasing frequency. Notably, the imaginary part of the dielectric permittivity for seawater may be higher than that for pool water, which in turn may be higher than that for fresh water.

Based on this plot, the GR conditions in pool water are expected around the 50 MHz resonant frequency. In agreement with theoretical discussion in Section 1, such a gradient resonance was indeed observed in surface wave-based underwater radio communication experiments. In these experiments the underwater divers were using conventional radios connected to plasmonic antennas operated at tunable carrier frequencies. The communication distance measured underwater near the water-air interface exhibited a clear resonance behavior (PGR) as a function of frequency, as measured by the ratio of communication distance to the skin depth δ in pool water, which is conventionally defined as

$$δ = \frac{{λ}_{0}}{2 π \sqrt{2 ( {( {ε}^{′2} + {ε}^{″2} )}^{1 / 2} - {ε}^{′} )}} \qquad ( EQN . 14 )$$

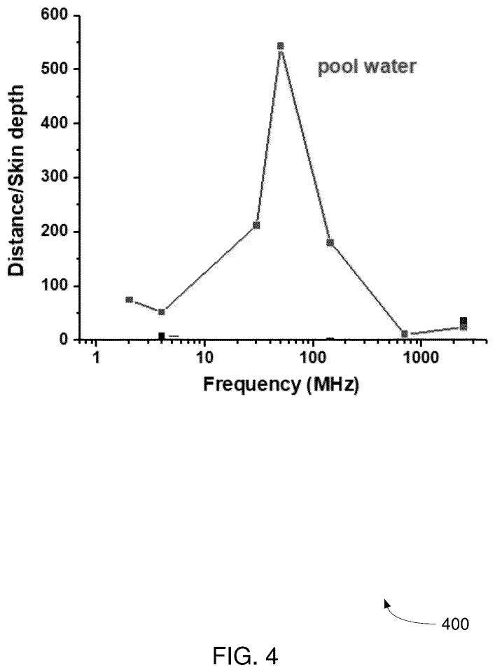

FIG. 4 shows a line graph 400 illustrating the propagation distance to the skin depth ratio for surface electromagnetic waves (plasmons) at the pool water-air interface in accordance with various aspects of the present disclosure. The line graph 400 may represent the relationship between the frequency of the electromagnetic waves and the ratio of the propagation distance to the skin depth in pool water.

The horizontal axis of the line graph 400 may represent the frequency of the electromagnetic waves, measured in megahertz (MHz). The scale of the horizontal axis may be logarithmic, ranging from 1 MHz to 1000 MHz, to effectively capture the wide range of frequencies under consideration. The vertical axis of the line graph 400 may represent the ratio of the propagation distance to the skin depth, a dimensionless quantity. The scale of the vertical axis may be linear, ranging from 0 to 600.

The data points in the line graph 400 may be marked with squares and connected by a line to indicate the trend. The graph may exhibit a pronounced peak at around 50 MHz, where the ratio of the propagation distance to the skin depth reaches its maximum value of approximately 500. This peak may indicate the occurrence of gradient resonance, where the propagation characteristics of the surface electromagnetic waves are significantly enhanced.

The line graph 400 may also show lower values of the ratio at frequencies below and above the resonant frequency of 50 MHz. For instance, at frequencies around 10 MHz and 1000 MHz, the ratio may drop to values below 100, indicating less efficient propagation of the surface waves.

Implementations described herein facilitate many applications of the PGR techniques in communication, sensing and imaging. For example, the PGR conditions may be used in mine and foreign objects detection on the water surface. In addition to extension of communication range, they may be used in biomedical imaging. The image contrast and spatial resolution of gradient resonance techniques may be on par with imaging based on nuclear magnetic resonance (NMR). On the other hand, unlike NMR, the PGR imaging may not be required to use very large magnetic fields which are necessary in NMR imaging, and which make it impractical in many field situations.

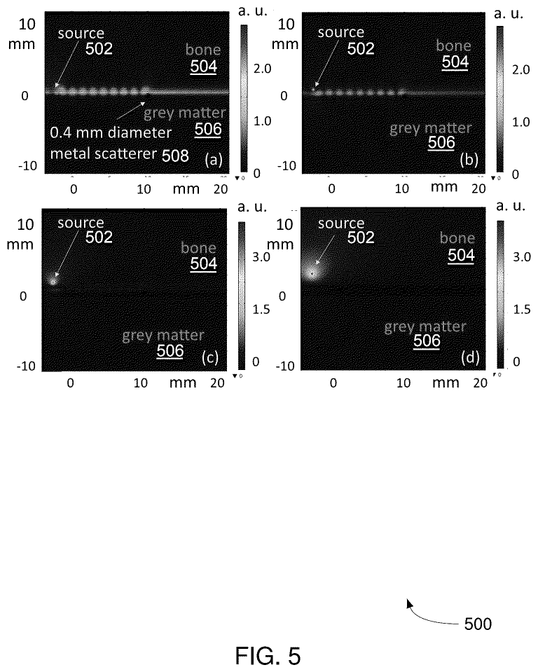

FIG. 5 shows modeling 500 of surface electromagnetic wave excitation at a boundary between the bone tissue and the gray matter in a human brane, while the excitation source was placed at different distances from the boundary in accordance with various aspects of the present disclosure. The PGR imaging and sensing techniques may be applied to such an interface, as well as many other tissue interfaces, in accordance with various aspects of the present disclosure. The modeling 500 may depict numerical simulations of surface electromagnetic wave propagation along an interface between the bone and gray matter in a human brain. As depicted in FIG. 5, the modeling 500 may include one or more of a source 502, bone 504, grey matter 506, a metal scatterer 508, and/or other components.

The source 502 may generate electromagnetic waves for gradient resonance in surface electromagnetic wave propagation. The source 502 may be designed to emit electromagnetic waves at specific frequencies that match the conditions for gradient resonance. These frequencies may be determined based on the dielectric properties of the medium through which the waves propagate. In some implementations, the source 502 may be adjustable to fine-tune the frequency of the emitted waves to achieve optimal resonance conditions. For example, the source 502 may be used in underwater communication systems where the salinity of the water affects the resonance frequency.

The bone 504 may interact with the electromagnetic waves to support gradient resonance. The bone 504 may have dielectric properties that influence the propagation of electromagnetic waves. These properties may include the permittivity and conductivity of the bone material. In some implementations, the interaction between the bone 504 and the electromagnetic waves may be analyzed to determine the presence of gradient resonance. For instance, the bone 504 may be part of a medical imaging system that uses gradient resonance to enhance image resolution.

The grey matter 506 may influence the propagation characteristics of the electromagnetic waves. The grey matter 506 may have a complex dielectric constant that varies with frequency, affecting how electromagnetic waves travel through it. This variation may be used in determining the conditions for gradient resonance. In some implementations, the grey matter 506 may be studied to understand its impact on surface wave propagation, which may be useful in biomedical imaging applications. For example, the grey matter 506 may be analyzed to detect abnormalities in brain tissue.

The metal scatterer 508 may affect the distribution and behavior of the electromagnetic waves. The metal scatterer 508 may introduce changes in the electromagnetic field, leading to scattering and reflection of the waves. These effects may be significant in environments where metal objects are present. In some implementations, the metal scatterer 508 may be used to study the impact of metallic structures on gradient resonance. For instance, the metal scatterer 508 may be part of an experimental setup to observe how metal objects influence the propagation of surface waves in a controlled environment.

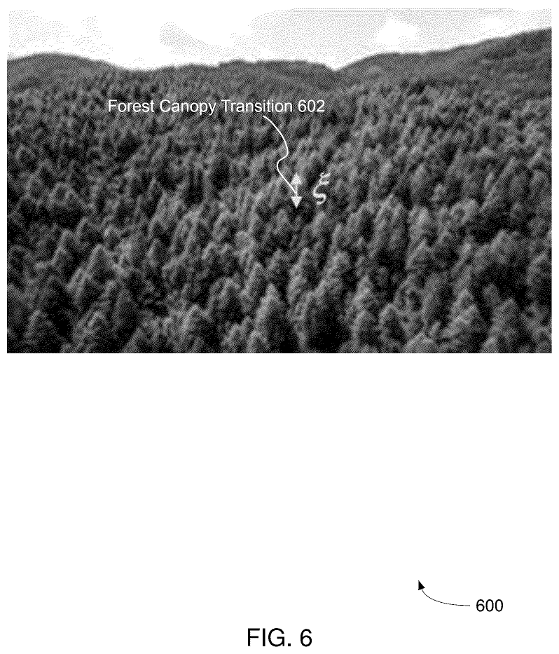

The PGR resonance may be applied in jungle communication and agricultural and hyperspectral RF imaging applications. The ability of surface waves to propagate on the surface of plant matter (see, e.g., FIG. 6) may be shown analytically by considering the properties of simple model in which the surface layer in the foliage transition layer is described by the following height-dependent dielectric properties:

$$ε = 1 + i {ε}^{″} ( z ) = 1 + i α z \qquad ( EQN . 15 )$$

which is has been justified by data. EQN. 15 leads to a simple rectangular effective potential at very small α<<1:

$$V ( z ) \approx - \frac{3}{4} {( \partial {ε}^{″} / \partial z )}^{2} = - \frac{3}{4} {α}^{2} \qquad ( EQN . 16 )$$

which describes an effective rectangular dielectric waveguide having average thickness ξ and effective dielectric permittivity

$${ε}_{eff} = ( 1 + \frac{3 {α}^{2} {λ}^{2}}{16 {π}^{2} {ξ}^{2}} ) + i \frac{α}{2} \qquad ( EQN . 17 )$$

On the other hand, as can be seen from EQN. 12, a PGR resonance in surface wave propagation will be observed around α≈1.

Since different plant matter and agricultural crops have different water content and different dielectric properties of their dry plant matter, the PGR conditions ε²=ε′²−ε″² will be observed at different RF resonance frequencies, which may enable the determination of crop health, presence of underground water beneath grassy areas, and many other sensing and imaging applications. A “hyperspectral” remote RF imaging scheme may be also applied, in which the RF spectrum of the surface will be measured at several frequencies, and a composite hyperspectral map of the land will be obtained.

FIG. 6 shows forest canopy transition 600 which supports techniques for gradient resonance in surface electromagnetic wave propagation for communication, sensing, and imaging in accordance with various aspects of the present disclosure. As depicted in FIG. 6, the forest canopy transition 600 may include one or more of a forest canopy transition 602 and/or other components. The forest canopy transition 602 may support techniques for gradient resonance in surface electromagnetic wave propagation. The forest canopy transition 602 may be characterized by a gradual change in dielectric properties along the z coordinate. This transition may create an effective waveguide for surface electromagnetic waves.

In some implementations, a plasmonic antenna may be configured to generate surface waves that propagate along the forest canopy transition 602, enabling interactions with the gradient resonance. The hyperspectral RF imaging may analyze the RF spectrum at multiple frequencies to construct a hyperspectral map, which may reveal variations in crop health determination and underground water detection. The dielectric property of the foliage may influence the resonance conditions, which may vary based on the water content and material composition of the plant matter.

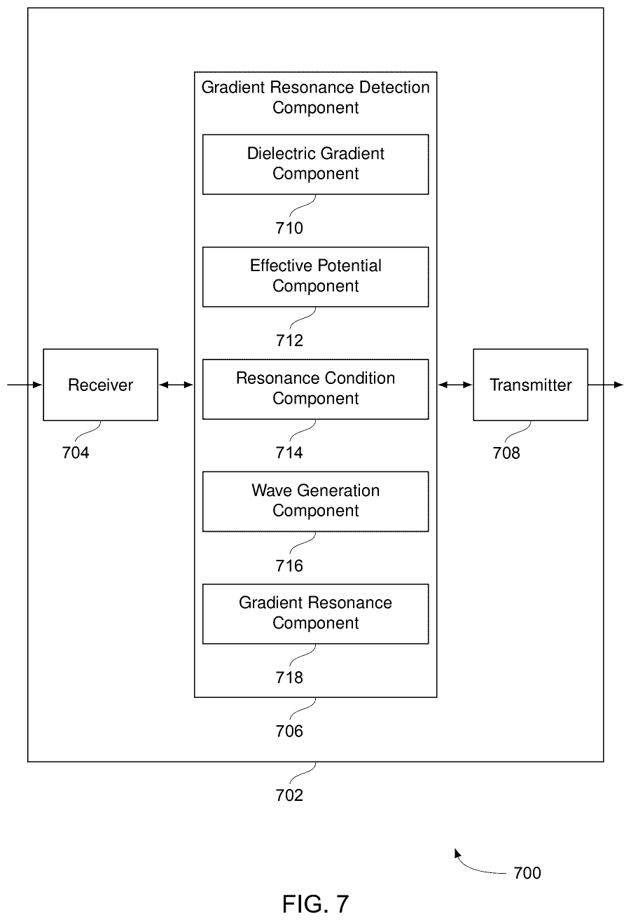

FIG. 7 shows a block diagram 700 of an apparatus 702 that supports gradient resonance in surface electromagnetic wave propagation for communication, sensing, and imaging in accordance with various aspects of the present disclosure. The apparatus 702 may include an input module 704, gradient resonance detection component 706, and an output module 708. The apparatus 702 may also include a processor. Each of these components may be in communication with one another (e.g., via one or more buses). In some cases, the apparatus 702 may be an example of a user terminal, a database server, or a system containing multiple computing devices.

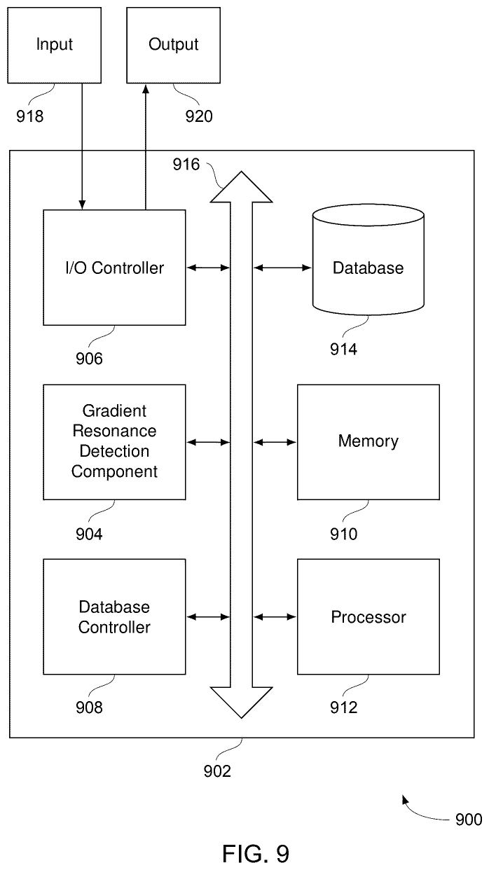

The input module 704 may manage input signals for the apparatus 702. For example, the input module 704 may identify input signals based on an interaction with a modem, a keyboard, a mouse, a touchscreen, or a similar device. These input signals may be associated with user input or processing at other components or devices. In some cases, the input module 704 may utilize an operating system such as iOS®, ANDROID®, MS-DOS®, MS-WINDOWS®, OS/2®, UNIX®, LINUX®, or another known operating system to handle input signals. The input module 704 may send aspects of these input signals to other components of the apparatus 702 for processing. For example, the input module 704 may transmit input signals to the gradient resonance detection component 706 to support face detection to address privacy in publishing image datasets. In some cases, the input module 704 may be a component of an input/output (I/O) controller 906 as described with reference to FIG. 9.

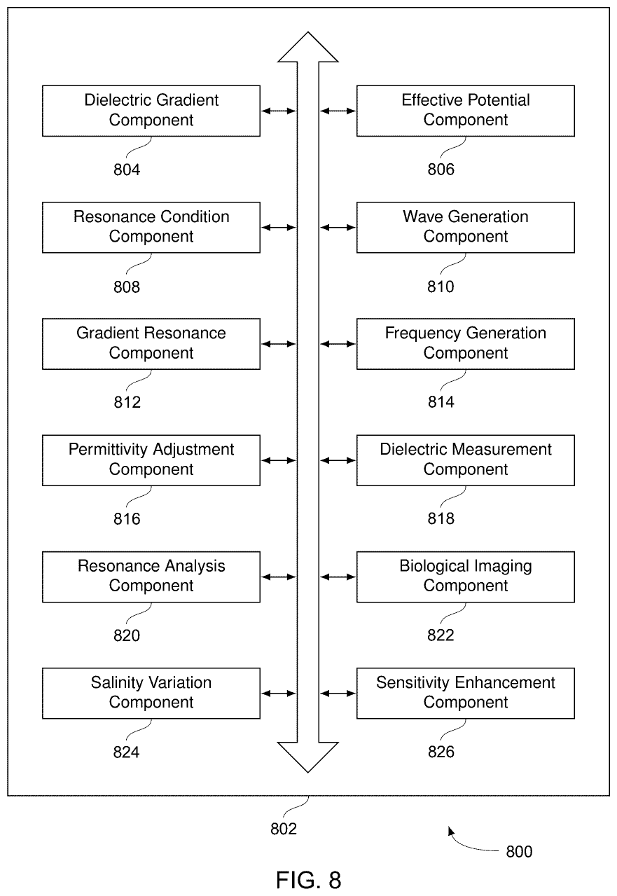

The gradient resonance detection component 706 may include one or more of a dielectric gradient component 710, an effective potential component 712, a resonance condition component 714, a wave generation component 716, a gradient resonance component 718, and/or other components. The gradient resonance detection component 706 may be an example of aspects of the gradient resonance detection component 802 or 904 described with reference to FIGS. 8 and 9.

The dielectric gradient component 710 may be configured as or otherwise support a means for obtaining a gradient of a dielectric permittivity of a medium along a propagation path of surface electromagnetic waves. The effective potential component 712 may be configured as or otherwise support a means for obtaining an effective potential based on the gradient of the dielectric permittivity and a second derivative of the dielectric permittivity. The resonance condition component 714 may be configured as or otherwise support a means for identifying a resonance condition in response to a real part of the dielectric permittivity squared approaching zero. The wave generation component 716 may be configured as or otherwise support a means for generating surface electromagnetic waves in response to the identified resonance condition. The gradient resonance component 718 may be configured as or otherwise support a means for detecting a gradient resonance in the surface electromagnetic waves.

The output module 708 may manage output signals for the apparatus 702. For example, the output module 708 may receive signals from other components of the apparatus 702, such as the gradient resonance detection component 706, and may transmit these signals to other components or devices. In some specific examples, the output module 708 may transmit output signals for display in a user interface, for storage in a database or data store, for further processing at a server or server cluster, or for any other processes at any number of devices or systems. In some cases, the output module 708 may be a component of an I/O controller 906 as described with reference to FIG. 9.

FIG. 8 shows a block diagram 800 of a gradient resonance detection component 802 that supports gradient resonance in surface electromagnetic wave propagation for communication, sensing, and imaging in accordance with various aspects of the present disclosure. The gradient resonance detection component 802 may be an example of aspects of a gradient resonance detection component 706, a gradient resonance detection component 904, or both, as described herein. The gradient resonance detection component 802, or various components thereof, may be an example of means for performing various aspects of gradient resonance in surface electromagnetic wave propagation for communication, sensing, and imaging as described herein. For example, the gradient resonance detection component 802 may include one or more of a dielectric gradient component 804, an effective potential component 806, a resonance condition component 808, a wave generation component 810, a gradient resonance component 812, a frequency generation component 814, a permittivity adjustment component 816, a dielectric measurement component 818, a resonance analysis component 820, a biological imaging component 822, a salinity variation component 824, a sensitivity enhancement component 826, and/or other components. Each of these components may communicate, directly or indirectly, with one another (e.g., via one or more buses).

The dielectric gradient component 804 may be configured as or otherwise support a means for obtaining a gradient of a dielectric permittivity of a medium along a propagation path of surface electromagnetic waves. In some implementations, the dielectric gradient component 804 may include a material with a gradual transition in dielectric properties, such as a water-air interface with varying salinity levels. In some implementations, the dielectric gradient component 804 may be designed to support surface electromagnetic waves that propagate along interfaces between different media, such as tissue boundaries in biological systems. In some implementations, the dielectric gradient component 804 may support the determination of resonance frequencies in water with specific salinity levels, such as pool water or seawater. In some implementations, the dielectric gradient component 804 may enable the determination of resonance features in agricultural environments, such as the top surface of a forest or a crop field. In some implementations, the dielectric gradient component 804 may support the propagation of surface waves along human tissue, such as the boundary between bone and gray matter. In some implementations, the dielectric gradient component 804 may support the propagation of surface waves in underwater environments, such as along the water-air interface in a pool.

The effective potential component 806 may be configured as or otherwise support a means for obtaining an effective potential based on the gradient of the dielectric permittivity and a second derivative of the dielectric permittivity. In some implementations, the effective potential component 806 may determine the effective potential in underwater communication experiments. In some implementations, the effective potential component 806 may be used to study the propagation of surface waves along human tissue boundaries.

The resonance condition component 808 may be configured as or otherwise support a means for identifying a resonance condition in response to a real part of the dielectric permittivity squared approaching zero. In some implementations, the resonance condition component 808 may identify the resonance condition when the real part of the dielectric permittivity squared is close to zero in water. The resonance condition component 808 may be used to detect resonance conditions in various materials with different dielectric properties.

The wave generation component 810 may be configured as or otherwise support a means for generating surface electromagnetic waves in response to the identified resonance condition. In some implementations, the surface electromagnetic waves may propagate along a water-air interface, where the dielectric properties of water may influence the wave behavior. In other implementations, the surface electromagnetic waves may propagate along tissue boundaries in biological systems, where variations in dielectric properties may create unique propagation characteristics.

The wave generation component 810 may include antennas that may operate at tunable carrier frequencies to match the resonance condition. In some implementations, these antennas may be harmonic antennas designed to emit RF signals at specific frequencies, such as 50 MHz, to align with the gradient resonance in pool water. In other implementations, the antennas may be configured to emit signals at frequencies suitable for observing resonance in agricultural or jungle environments.

The wave generation component 810 may be designed to support communication, sensing, or imaging applications based on the generated surface electromagnetic waves. In some implementations, the generated waves may enable underwater communication between divers equipped with radios connected to the component. In other implementations, the generated waves may facilitate imaging of human tissue boundaries or sensing of water content in agricultural crops.

The gradient resonance component 812 may be configured as or otherwise support a means for detecting a gradient resonance in the surface electromagnetic waves. In some implementations, the gradient resonance component 812 may detect resonance conditions in water with varying salinity levels. The gradient resonance component 812 may be used to identify resonance at specific frequencies, such as 50 MHz in pool water.

In some examples, the frequency generation component 814 may be configured as or otherwise support a means for generating surface electromagnetic waves at a frequency corresponding to the identified resonance condition. In some implementations, the frequency generation component 814 may be adjusted to operate at different frequencies to match various resonance conditions in different media. The frequency generation component 814 may include tunable elements that may allow for precise control over the generated frequency.

In some examples, the permittivity adjustment component 816 may be configured as or otherwise support a means for adapting the dielectric permittivity of the medium to achieve the gradient of the dielectric permittivity along the propagation path. In some implementations, the adaptation may involve modifying the salinity of water to influence its dielectric properties, as observed in experiments with pool water. In some implementations, the adaptation may include introducing a gradual transition layer between two media to create a controlled gradient in the dielectric permittivity.

In some examples, the dielectric measurement component 818 may be configured as or otherwise support a means for measuring the dielectric properties of the medium to determine the gradient of the dielectric permittivity. In some implementations, the dielectric measurement component 818 may utilize sensors to measure the dielectric properties at various points along the propagation path. The dielectric measurement component 818 may include a data processing unit to analyze the measured dielectric properties and determine the gradient. In some implementations, the dielectric measurement component 818 may be integrated with other components to enhance the accuracy of the measurements.

In some examples, the resonance analysis component 820 may be configured as or otherwise support a means for analyzing the detected gradient resonance to determine the communication distance of the surface electromagnetic waves. In some implementations, the resonance analysis component 820 may utilize data from underwater radio communication experiments to determine the communication distance. In some implementations, the resonance analysis component 820 may analyze the ratio of communication distance to the skin depth in various water salinity levels.

In some examples, the resonance analysis component 820 may be configured to analyze the resonance behavior as a function of frequency. In some implementations, the resonance analysis component 820 may use theoretical models to determine the resonance frequency in different media. In some implementations, the resonance analysis component 820 may compare experimental data with theoretical predictions to determine the accuracy of the resonance condition.

In some examples, the resonance analysis component 820 may be configured to identify the resonance condition in various environments. In some implementations, the resonance analysis component 820 may analyze the dielectric properties of different tissues to determine the resonance condition. In some implementations, the resonance analysis component 820 may use numerical simulations to determine the resonance behavior in complex media.

In some examples, the biological imaging component 822 may be configured as or otherwise support a means for using the detected gradient resonance to perform imaging of biological tissues. In some implementations, the biological imaging component 822 may use the resonance condition to identify variations in tissue composition based on the dielectric properties of the medium. In some implementations, the biological imaging component 822 may detect boundaries between different tissue types, such as bone and gray matter, by analyzing the surface wave propagation characteristics.

In some implementations, the biological imaging component 822 may be configured to operate at specific frequencies corresponding to the resonance condition to achieve imaging sensitivity. In some implementations, the biological imaging component 822 may analyze the dielectric properties of water content in tissues to identify abnormalities. In some implementations, the biological imaging component 822 may detect subtle changes in tissue boundaries by observing shifts in the resonance frequency.

In some examples, the salinity variation component 824 may be configured as or otherwise support a means for obtaining the gradient of the dielectric permittivity by varying the salinity of water in the medium. In some implementations, the salinity variation component 824 may adjust the salinity levels to specific values to achieve desired dielectric properties. In some implementations, the salinity variation component 824 may include sensors to monitor the salinity levels in real-time.

In some examples, the sensitivity enhancement component 826 may be configured as or otherwise support a means for enhancing the sensitivity of RF imaging techniques using the detected gradient resonance. In some implementations, the sensitivity enhancement component 826 may be used to detect small variations in tissue composition. The sensitivity enhancement component 826 may also be applied to agricultural sensing to distinguish between different crop health conditions. In some implementations, the sensitivity enhancement component 826 may be used in conjunction with other imaging techniques to achieve higher resolution.

FIG. 9 shows a diagram of a system 900 including a device 902 that supports gradient resonance in surface electromagnetic wave propagation for communication, sensing, and imaging in accordance with aspects of the present disclosure. The device 902 may be an example of or include the components of a database server or an apparatus 702 as described herein. The device 902 may include components for bi-directional data communications including components for transmitting and receiving communications, including a gradient resonance detection component 904, an I/O controller 906, a database controller 908, memory 910, a processor 912, and a database 914. These components may be in electronic communication via one or more buses (e.g., bus 916).

The gradient resonance detection component 904 may be an example of a gradient resonance detection component 706 or 802 as described herein. For example, the gradient resonance detection component 904 may perform any of the methods or processes described above with reference to FIGS. 7 and 8. In some cases, the gradient resonance detection component 904 may be implemented in hardware, software executed by a processor, firmware, or any combination thereof.

The I/O controller 906 may manage input signals 918 and output signals 920 for the device 902. The I/O controller 906 may also manage peripherals not integrated into the device 902. In some cases, the I/O controller 906 may represent a physical connection or port to an external peripheral. In some cases, the I/O controller 906 may utilize an operating system such as iOS®, ANDROID®, MS-DOS®, MS-WINDOWS®, OS/2®, UNIX®, LINUX®, or another known operating system. In other cases, the I/O controller 906 may represent or interact with a modem, a keyboard, a mouse, a touchscreen, or a similar device. In some cases, the I/O controller 906 may be implemented as part of a processor. In some cases, a user may interact with the device 902 via the I/O controller 906 or via hardware components controlled by the I/O controller 906.

The database controller 908 may manage data storage and processing in a database 914. In some cases, a user may interact with the database controller 908. In other cases, the database controller 908 may operate automatically without user interaction. The database 914 may be an example of a single database, a distributed database, multiple distributed databases, a data store, a data lake, or an emergency backup database.

Memory 910 may include random-access memory (RAM) and read-only memory (ROM). The memory 910 may store computer-readable, computer-executable software including instructions that, when executed, cause the processor to perform various functions described herein. In some cases, the memory 910 may contain, among other things, a basic input/output system (BIOS) which may control basic hardware or software operation such as the interaction with peripheral components or devices.

The processor 912 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, the processor 912 may be configured to operate a memory array using a memory controller. In other cases, a memory controller may be integrated into the processor 912. The processor 912 may be configured to execute computer-readable instructions stored in a memory 910 to perform various functions (e.g., functions or tasks supporting gradient resonance in surface electromagnetic wave propagation for communication, sensing, and imaging).

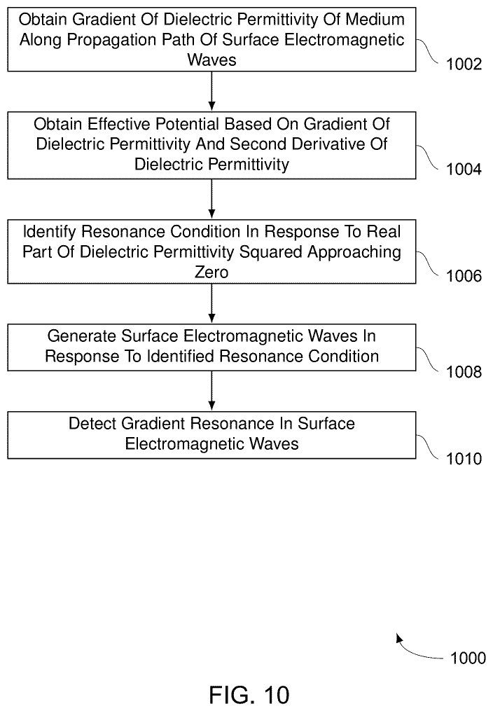

FIG. 10 shows a flowchart illustrating a method 1000 that supports gradient resonance in surface electromagnetic wave propagation for communication, sensing, and imaging in accordance with various aspects of the present disclosure. The operations of the method 1000 may be implemented by one or more components of a networked computing system as described herein. For example, the operations of the method 1000 may be performed by a gradient resonance detection component as described with reference to FIGS. 7 through 9. In some examples, one or more components of a networked computing system may execute a set of instructions to control the functional elements of the component(s) to perform the described functions. Additionally or alternatively, the one or more components of a networked computing system may perform aspects of the described functions using special-purpose hardware.

At 1002, the method 1000 may include obtaining a gradient of a dielectric permittivity of a medium along a propagation path of surface electromagnetic waves. The operations of 1002 may be performed in accordance with examples as disclosed herein. In some examples, aspects of the operations of 1002 may be performed by a dielectric gradient component 804 as described with reference to FIG. 8.

At 1004, the method 1000 may include obtaining an effective potential based on the gradient of the dielectric permittivity and a second derivative of the dielectric permittivity. The operations of 1004 may be performed in accordance with examples as disclosed herein. In some examples, aspects of the operations of 1004 may be performed by an effective potential component 806 as described with reference to FIG. 8.

At 1006, the method 1000 may include identifying a resonance condition in response to a real part of the dielectric permittivity squared approaching zero. The operations of 1006 may be performed in accordance with examples as disclosed herein. In some examples, aspects of the operations of 1006 may be performed by a resonance condition component 808 as described with reference to FIG. 8.

At 1008, the method 1000 may include generating surface electromagnetic waves in response to the identified resonance condition. The operations of 1008 may be performed in accordance with examples as disclosed herein. In some examples, aspects of the operations of 1008 may be performed by a wave generation component 810 as described with reference to FIG. 8.

At 1010, the method 1000 may include detecting a gradient resonance in the surface electromagnetic waves. The operations of 1010 may be performed in accordance with examples as disclosed herein. In some examples, aspects of the operations of 1010 may be performed by a gradient resonance component 812 as described with reference to FIG. 8.

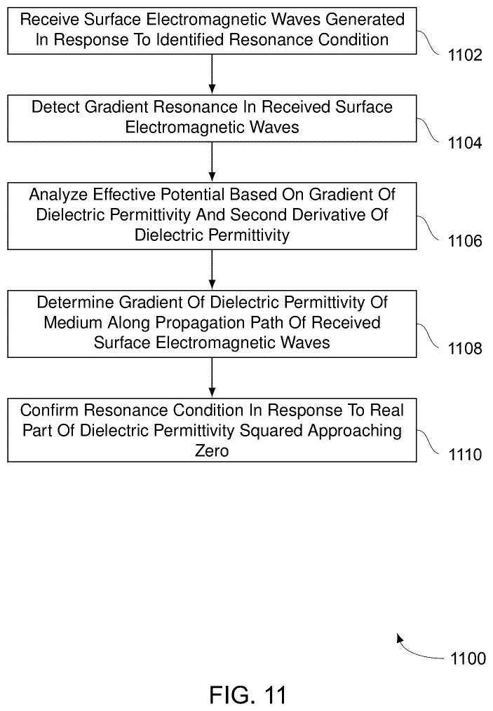

FIG. 11 shows a flowchart illustrating a method 1100 that supports gradient resonance in surface electromagnetic wave propagation for communication, sensing, and imaging in accordance with various aspects of the present disclosure. The operations of the method 1100 may be implemented by one or more components of a networked computing system as described herein. For example, the operations of the method 1100 may be performed by a gradient resonance detection component as described with reference to FIGS. 7 through 9. In some examples, one or more components of a networked computing system may execute a set of instructions to control the functional elements of the component(s) to perform the described functions. Additionally or alternatively, the one or more components of a networked computing system may perform aspects of the described functions using special-purpose hardware.

At 1102, the method 1100 may include receiving surface electromagnetic waves generated in response to an identified resonance condition. The operations of 1102 may be performed in accordance with examples as disclosed herein. In some examples, aspects of the operations of 1102 may be performed by a wave generation component 810 as described with reference to FIG. 8.

At 1104, the method 1100 may include detecting a gradient resonance in the received surface electromagnetic waves. The operations of 1104 may be performed in accordance with examples as disclosed herein. In some examples, aspects of the operations of 1104 may be performed by a gradient resonance component 812 as described with reference to FIG. 8.

At 1106, the method 1100 may include analyzing the effective potential based on the gradient of the dielectric permittivity and a second derivative of the dielectric permittivity. The operations of 1106 may be performed in accordance with examples as disclosed herein. In some examples, aspects of the operations of 1106 may be performed by an effective potential component 806 as described with reference to FIG. 8.

At 1108, the method 1100 may include determining the gradient of the dielectric permittivity of a medium along a propagation path of the received surface electromagnetic waves. The operations of 1108 may be performed in accordance with examples as disclosed herein. In some examples, aspects of the operations of 1108 may be performed by a dielectric gradient component 804 as described with reference to FIG. 8.

At 1110, the method 1100 may include confirming the resonance condition in response to a real part of the dielectric permittivity squared approaching zero. The operations of 1110 may be performed in accordance with examples as disclosed herein. In some examples, aspects of the operations of 1110 may be performed by a resonance condition component 808 as described with reference to FIG. 8.

It should be noted that the methods described herein describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for communication, sensing, and imaging based on gradient resonance in surface electromagnetic waves, comprising:
obtaining a gradient of a dielectric permittivity of a medium along a propagation path of surface electromagnetic waves;
obtaining an effective potential based on the gradient of the dielectric permittivity and a second derivative of the dielectric permittivity;
identifying a resonance condition in response to a real part of the dielectric permittivity squared approaching zero;
generating surface electromagnetic waves in response to the identified resonance condition; and
detecting a gradient resonance in the surface electromagnetic waves.

2. The method of claim 1, further comprising generating surface electromagnetic waves at a frequency corresponding to the identified resonance condition.

3. The method of claim 1, further comprising adapting the dielectric permittivity of the medium to achieve the gradient of the dielectric permittivity along the propagation path.

4. The method of claim 1, further comprising measuring the dielectric properties of the medium to determine the gradient of the dielectric permittivity.

5. The method of claim 1, further comprising analyzing the detected gradient resonance to determine the communication distance of the surface electromagnetic waves.

6. The method of claim 1, further comprising using the detected gradient resonance to perform imaging of biological tissues.

7. The method of claim 1, wherein the gradient of the dielectric permittivity is obtained by varying the salinity of water in the medium.

8. The method of claim 1, wherein the effective potential is determined by the first derivative of the dielectric permittivity along the propagation path.

9. The method of claim 1, wherein the surface electromagnetic waves are generated at a frequency range between 1 MHz and 1000 MHz.

10. The method of claim 1, wherein the detected gradient resonance is used to enhance the sensitivity of RF imaging techniques.

11. The method of claim 1, wherein the surface electromagnetic waves are used to detect the presence of underground water beneath agricultural fields.

12. The method of claim 1, wherein the gradient resonance is identified in response to a change in the dielectric properties of the medium.

13. A system configured for communication, sensing, and imaging based on gradient resonance in surface electromagnetic waves, comprising:
a processor;
memory coupled with the processor; and
instructions stored in the memory and executable by the processor to cause the system to:
obtain a gradient of a dielectric permittivity of a medium along a propagation path of surface electromagnetic waves;
obtain an effective potential based on the gradient of the dielectric permittivity and a second derivative of the dielectric permittivity;
identify a resonance condition in response to a real part of the dielectric permittivity squared approaching zero;
generate surface electromagnetic waves in response to the identified resonance condition; and
detect a gradient resonance in the surface electromagnetic waves.

14. The system of claim 13, wherein the instructions are further executable by the processor to cause the system to: generate surface electromagnetic waves at a frequency corresponding to the identified resonance condition.

15. The system of claim 13, wherein the instructions are further executable by the processor to cause the system to: adapt the dielectric permittivity of the medium to achieve the gradient of the dielectric permittivity along the propagation path.

16. The system of claim 13, wherein the instructions are further executable by the processor to cause the system to: measure the dielectric properties of the medium to determine the gradient of the dielectric permittivity.

17. The system of claim 13, wherein the instructions are further executable by the processor to cause the system to: analyze the detected gradient resonance to determine the communication distance of the surface electromagnetic waves.

18. The system of claim 13, wherein the instructions are further executable by the processor to cause the system to: use the detected gradient resonance to perform imaging of biological tissues.

19. The system of claim 13, wherein the gradient of the dielectric permittivity is obtained by varying the salinity of water in the medium.

20. A non-transitory computer-readable medium storing code for communication, sensing, and imaging based on gradient resonance in surface electromagnetic waves, the code comprising instructions executable by a processor to:
obtain a gradient of a dielectric permittivity of a medium along a propagation path of surface electromagnetic waves;
obtain an effective potential based on the gradient of the dielectric permittivity and a second derivative of the dielectric permittivity;
identify a resonance condition in response to a real part of the dielectric permittivity squared approaching zero;
generate surface electromagnetic waves in response to the identified resonance condition; and
detect a gradient resonance in the surface electromagnetic waves.
